(19) United States
(12) Patent Application Publication
(10) Pub. No.: US 2025/0283862 A1
(43) Pub. Date: Sep. 11, 2025

(54) METHOD FOR OPERATING A GAS MEASURING SYSTEM AND CORRESPONDING GAS MEASURING SYSTEM

(71) Applicant: Dräger Safety AG & Co. KGaA, Lübeck (DE)

(72) Inventors: Marie-Isabell Mattern-Frühwald, Lübeck (DE); Malte Baesler, Lübeck (DE)

(21) Appl. No.: 19/073,764

(22) Filed: Mar. 7, 2025

(30) Foreign Application Priority Data
Mar. 11, 2024 (DE) 102024106856.6

(51) Int. Cl. G01N 33/00; G01N 33/497; G08B 21/18

(52) U.S. Cl. CPC G01N 33/007; G01N 33/0031; G01N 33/0063; G01N 33/4972; G08B 21/182

(57) ABSTRACT

A method for operating a gas measuring system is provided. The method may comprise the steps of: storing a gas measuring device in a storage environment in a first operating state; determining a concentration of a harmful gas in the storage environment by means of an evaluation unit; determining a concentration of the target gas in a measurement environment by means of the evaluation unit in a second operating state, taking into account the concentration of the harmful gas, and outputting the concentration of the target gas; and/or comparing information correlating with the concentration of the harmful gas with a predetermined threshold value by means of the evaluation unit and issuing a warning in a third operating state if the correlating information exceeds the predetermined threshold value. Furthermore, a corresponding gas measuring system is provided.

### CROSS-REFERENCE TO RELATED APPLICATIONS

This application claims the priority of German Patent Application No. 102024106856.6, filed on Mar. 11, 2024, and titled “METHOD FOR OPERATING A GAS MEASURING SYSTEM AND CORRESPONDING GAS MEASURING SYSTEM,” which is hereby incorporated by reference in its entirety for all nonlimiting purposes.

### BACKGROUND

The present disclosure relates to a method for operating a gas measuring system and to a corresponding gas measuring system.

Gas measuring devices having one or more gas sensors, in particular having one or more electrochemical gas sensors, such as electrochemical alcohol sensors, can be reversibly or irreversibly damaged by storage in an environment containing harmful gas (storage environment), thus affecting their measuring behavior.

An example of such a gas measuring device is a breathalyzer. In a gas measuring device, in particular a breathalyzer, a reduction in the sensitivity of the gas sensor can occur, for example during long-term storage (e.g., one or more days and/or weeks) in a storage environment with, for example, alcohols as the harmful gas. This reduction may persist for several days even after the gas measuring device has been removed from the storage environment. Storing the gas measuring device in an environment containing a harmful gas will therefore impair its measuring behavior. However, a user of the gas measuring device is unable to determine whether such an impairment has occurred.

Alcohol can, for example, occur as a harmful gas through the use of disinfectants (e.g., hand disinfectants) or other solvents in the storage environment. Storing disinfectant containers or solvent containers in the storage environment can also lead to the release of harmful gas such as alcohol.

The above-described problem of the occurrence of harmful gases and their effect on the measuring behavior of the gas measuring device affects in particular gas measuring devices without a diffusion barrier to the environment, as is the case, for example, with many of the currently commercially available breathalyzers customary in the market.

A solution to the above problem is currently not known. DE 10 2022 108 432 A1 simply discloses that the problem can be circumvented by hermetically sealing a sensor of a gas measuring device during storage so that the potentially harmful storage environment cannot affect the sensor. This method therefore requires complex and expensive mechanical encapsulation of the sensor.

US 2024/0 013 647 A1 discloses a system and a method for detecting gas while simultaneously correcting a poison level of a gas sensor. The gas sensor comprises a gas sensor material configured to come into contact with a fluid sample; and a measuring circuit configured to provide first and second dielectric excitation of the gas sensor material for a first and second set of frequencies, respectively, while the gas sensor material is in contact with the fluid sample. On the basis of a measured response of the gas sensor material to the first and second dielectric excitation at each of the first and second frequency sets, a poison level of the gas sensor material is corrected.

It is therefore an object of the present disclosure to provide a method for operating a gas measuring system and a corresponding gas measuring system which does not have the above-mentioned disadvantages or only has them to a reduced extent.

### BRIEF DESCRIPTION OF THE DRAWINGS

FIG. 1 shows an exemplary embodiment of a schematically illustrated gas measuring system according to the disclosure.

FIG. 2 shows a further exemplary embodiment of a schematically illustrated gas measuring system according to the disclosure.

FIG. 3 shows a schematic flow diagram according to one exemplary embodiment of a method according to the disclosure.

FIG. 4 shows a schematic flow diagram according to a further exemplary embodiment of a method according to the disclosure.

FIG. 5 shows a schematic flow diagram according to yet a further exemplary embodiment of a method according to the disclosure.

FIG. 6 shows example time curves of the concentration of harmful gas in a storage environment and of a state of poisoning.

### DETAILED DESCRIPTION

A method for operating a gas measuring system is provided according to the disclosure, the method comprising the steps of: storing a gas measuring device in a storage environment in a first operating state; determining a concentration of a harmful gas in the storage environment by means of an evaluation unit; determining a concentration of the target gas in a measurement environment by means of the evaluation unit in a second operating state, taking into account the concentration of the harmful gas, and outputting the concentration of the target gas; and/or comparing information correlating with the concentration of the harmful gas with a predetermined threshold value by means of the evaluation unit and issuing a warning in a third operating state if the correlating information exceeds the predetermined threshold value.

In this way, the effect of the storage environment on the measuring behavior of the gas measuring device can be identified and a user of the gas measuring device can be warned by issuing the warning (immediately or in a delayed manner, i.e., downstream) that the measuring behavior has deteriorated. In addition or as an alternative to issuing the warning, the effect of the concentration and/or quantity of the harmful gas on the calculated concentration of the target gas can be at least partially compensated in order to output a correspondingly corrected concentration of the target gas. In both cases, it is thus possible to establish a (qualitative and/or quantitative) effect of the storage environment on the measuring behavior of the gas measuring device and to take this into account when operating the gas measuring device.

A storage environment is understood to mean an environment in which a gas measuring device can be stored in a first operating state and in which a harmful gas may be present.

The first operating state is an operating state of the gas measuring device in which a concentration of the harmful gas is measured.

The second operating state is an operating state of the gas measuring device in which a concentration of the target gas is measured.

The third operating state is an operating state of the gas measuring device in which a warning is issued to a user of the gas measuring device.

The first operating state and the third operating state can occur simultaneously or at different times. The second operating state and the third operating state can occur simultaneously or at different times. The first operating state and the second operating state occur at different times.

A harmful gas is understood to mean a substance that can affect a measuring behavior of the gas measuring device, in particular through prolonged exposure thereto. This can be a target gas or a different substance. For example, the harmful gas may be an alcohol (e.g., ethanol, methanol, 1-(iso) propanol and/or 2-(iso) propanol).

A target gas is understood to be a gas or gas mixture whose concentration in a measurement environment of the gas measuring device is to be determined and output. This may also be an alcohol (e.g., ethanol, methanol, 1-(iso) propanol and/or 2-(iso) propanol).

The case that both the harmful gas and the target gas can be an alcohol may occur, for example, if the gas measuring device is designed as a breathalyzer which is stored in a storage environment that potentially contains alcohol. Alcohols may be present in the storage environment, for example as a result of disinfectants or cleaning agents (e.g., windshield washer fluid or cockpit cleaner for cars). An alcohol in the form of a harmful gas is present, for example, in a concentration in the percent range, while an alcohol in the form of a target gas can, for example, be present in a concentration in the per mil range.

The measurement environment can be a region immediately surrounding the gas measuring device, for example an atmosphere surrounding the gas measuring device. Additionally or alternatively, the measurement environment can be a region indirectly surrounding the gas measuring device, for example a gas or gas mixture that is supplied to the gas measuring device via a sample supplying element and which may be different from the region immediately surrounding it. When measuring breathing gas, the measurement environment is, for example, a breathing gas sample, which can be supplied to the gas measuring device using a mouthpiece or funnel, for example.

An evaluation unit is understood to mean a component of a gas measuring system designed to use suitable hardware and/or software to carry out signal inputting, data processing and signal outputting steps. The evaluation unit can be configured to carry out some or all of the steps of the method according to the disclosure. The evaluation unit can be designed, for example, as a processor or as a microcontroller. The evaluation unit can be provided as a component of the gas measuring device or as a separate component. If the gas measuring device and the evaluation unit are physically separate components, it is preferable for a data connection to be providable therebetween, for example via a wireless interface.

The warning can be issued by means of an alarm unit, wherein the warning can be issued optically and/or acoustically. The warning in the third operating state can, for example, be issued when the device is transferred to the second operating state or is in this state or even during storage (i.e., in the first operating state).

The steps “storing the gas measuring device in the storage environment in the first operating state” and “determining a concentration of a harmful gas in the storage environment by means of an evaluation unit” preferably take place simultaneously, wherein the step “determining the concentration of the target gas in the measurement environment by means of the evaluation unit in the second operating state” taking into account the concentration of the harmful gas and “outputting the concentration of the target gas” and/or the step “comparing the information correlating with the concentration of the harmful gas with the predetermined threshold value by means of the evaluation unit and issuing the warning in a third operating state” if the correlating information exceeds the predetermined threshold value preferably take place at different times.

Preferably, the step “determining the concentration of the harmful gas in the storage environment” is repeated, in particular repeatedly carried out periodically in the first operating state.

In this way, in the first operating state, a plurality of time-resolved measurement signals, which correlate with the concentration of the harmful gas at the corresponding measurement times, can be obtained and made available for (simultaneous or subsequent) processing by the evaluation unit.

For example, in the first operating state, it is possible to regularly provide measurement signals, i.e., to repeatedly provide them periodically, at an interval of, for example, 1 minute, which signals correlate with the concentration of the harmful gas in the storage environment and thus provide measured values of the concentration of the harmful gas, and to evaluate them by means of the evaluation unit.

Preferably, the method further comprises the step of determining whether the concentration of the harmful gas and/or a quantity of the harmful gas exceeds the predetermined threshold value, wherein this method step is carried out by the evaluation unit. Furthermore, the method preferably comprises the step of issuing the warning if the concentration and/or quantity of the harmful gas exceeds or has exceeded the predetermined threshold value.

The concentration and/or the quantity of the harmful gas are thus examples of information that correlates with the concentration of the harmful gas.

In this way, the process of issuing the warning can be adjusted in such a way that a check is made to see whether a concentration of the harmful gas has exceeded a predetermined threshold value and/or whether a quantity of the harmful gas has exceeded a predetermined threshold value. Depending on the characteristics of the gas measuring system to be monitored, the process of issuing a warning can be adapted to the measuring behavior of the gas measuring device.

The warning can be issued immediately as soon as the entry condition is met or in a step that takes place at a different time, for example as soon as a user switches on the gas measuring device (transition from the first operating state to the second operating state).

Preferably, a first gas sensor is provided for determining the concentration of the harmful gas in the storage environment, wherein a second (i.e., separate) gas sensor is provided for determining the concentration of the target gas in the measurement environment.

In this way, the measurement of the harmful gas and the measurement of the target gas can be carried out by separate gas sensors. In this respect, the measuring behavior of each of the gas sensors can be optimized for measuring the harmful gas and measuring the target gas, the relevant environmental conditions, such as high concentrations of harmful gas and low concentrations of target gas, and a corresponding sampling method in each case. Furthermore, it may be possible to reduce the energy consumption of the gas measuring system on average over time since the storage environment can be monitored only by means of the first gas sensor, while the second gas sensor is not operated in the first operating state.

It is preferred that the first gas sensor and/or the second gas sensor is/are each designed as an electrochemical gas sensor.

An electrochemical gas sensor is understood to mean an electrochemical cell which is configured to detect at least one gaseous substance, in particular the harmful gas and/or the target gas, in a gas or gas mixture, in particular in the storage environment or measurement environment, and to provide a number of measurement signals corresponding to the concentration of the substance.

Preferably, the electrochemical gas sensor comprises at least one measuring electrode and a counter electrode, further preferably at least one reference electrode. It is possible for further electrodes to be present. Preferably, the electrochemical gas sensor comprises an acidic liquid electrolyte which is in contact with at least some of the electrodes. The electrochemical gas sensor can be designed as a fuel cell.

Particularly preferably, the first gas sensor is designed as a 3-electrode sensor. Additionally or alternatively, it is preferred that the second gas sensor is designed as a 2-electrode sensor. It is preferred that the 3-electrode sensor operates according to an amperometric measuring principle and that the 2-electrode sensor operates according to a coulometric measuring principle.

It is preferred that the first gas sensor has long-term resistance to the harmful gas and that the second gas sensor is particularly sensitive to the target gas.

It is preferred that the first gas sensor is configured for passive sampling and/or that the second gas sensor is configured for active sampling, for example by means of a pump apparatus.

If one of the two gas sensors is designed as a 3-electrode sensor, it is preferable for the gas measuring device to further comprise a potentiostat circuit.

The gas measuring device can comprise further components, for example a sampling system comprising a lifting magnet and a bellows.

Preferably, the step of determining the concentration of the target gas in the measurement environment in the second operating state, taking into account the concentration of the harmful gas, comprises the step of correcting a sensitivity of the second gas sensor by means of the evaluation unit.

In this way, a corrected concentration of the target gas can be output particularly easily.

Preferably, the step of correcting the sensitivity of the second gas sensor comprises the additional step of determining a current state of poisoning and/or a current state of recovery of the second gas sensor by means of the evaluation unit.

In this way, the dynamic effect of the harmful gas on the measuring behavior of the second gas sensor can be taken into account.

The state of poisoning refers to an adverse effect of the harmful gas on the sensitivity of the second gas sensor. The state of recovery refers to the reduction in the effect of the harmful gas on the sensitivity of the second gas sensor.

It is particularly preferred to take into account both the state of poisoning and the state of recovery since it is possible that the dynamic behavior of the state of poisoning and the dynamic behavior of the state of recovery have different time constants.

According to the disclosure, a gas measuring system is also provided.

The gas measuring system has a first gas sensor, which is configured to provide a first measurement signal correlating with a concentration of a harmful gas in a storage environment of the first gas sensor present gas. The gas measuring system further comprises a second gas sensor, which is configured to provide a second measurement signal correlating with a concentration of a target gas in a measurement environment of the second gas sensor present gas. The gas measuring system further comprises an evaluation unit, which is configured to receive the first measurement signal and the second measurement signal and to carry out some or all of the steps of the above-described method according to the disclosure.

The gas measuring system has similar advantages and effects to the above-described method according to the disclosure. All features and preferred embodiments disclosed in connection with the method are also deemed to be disclosed in connection with the gas measuring system, and vice versa.

The first gas sensor, the second gas sensor and the evaluation unit may be provided in a common device or in different devices. For example, the first gas sensor and the evaluation unit may be provided in a first device and the second gas sensor in a second device. In another example, the second gas sensor and the evaluation unit may be provided in a first device and the first gas sensor in a second device. In a further example, the second gas sensor and the evaluation unit may be provided in a first device and the first gas sensor and a further or additional evaluation unit in a second device.

Preferably, the gas measuring system comprises a breathalyzer, which is configured to determine a concentration of alcohol as a target gas in a measurement environment of the breathalyzer, in particular in a breathing gas sample, wherein the breathalyzer comprises the second gas sensor. The breathalyzer may also comprise the first gas sensor, although this is not necessary. The breathalyzer may also comprise the evaluation unit, although this is also not necessary.

For example, the breathalyzer can be a breath alcohol measuring device.

The breathalyzer, in particular the breath alcohol measuring device, can have an active sampling system such as a pump apparatus or pump unit in order to supply a volume of a breathing gas sample, preferably a defined volume, to the second gas sensor and to determine the concentration of alcohol as a target gas in the measurement environment, in particular in the breathing gas sample.

Preferably, the gas measuring system comprises a monitoring device, which is configured to determine the concentration of the harmful gas in the storage environment. The monitoring device comprises the first gas sensor. A monitoring device is therefore a device that comprises the first gas sensor for monitoring the concentration of the harmful gas in the storage environment.

In this preferred embodiment, the gas measuring system thus comprises at least two separate devices, specifically the breathalyzer and the monitoring device. It is preferred that the monitoring device further comprises the evaluation unit, although this is not necessary. A data connection can be provided between the breathalyzer and the monitoring device via a data interface, for example via a wireless connection.

In this way, a gas measuring system according to the disclosure can be provided by retrofitting a known breathalyzer by providing the monitoring device as an additional component of the gas measuring system. The functionality of existing breathalyzers can thus be improved.

Preferably, the breathalyzer and/or the monitoring device comprises an alarm unit for optically and/or acoustically issuing the warning. The alarm unit can, for example, be designed as a display and/or a loudspeaker. It is preferred that the alarm unit is designed as a component of the monitoring device, but this is not necessary.

All features disclosed herein may be combined with one another as desired, provided this does not relate to alternatives and is not contradictory.

These and other features and advantageous embodiments of the disclosure can be found in the following description of the drawings. In the drawings:

FIG. 1 shows an exemplary embodiment of a schematically illustrated gas measuring system according to the disclosure,

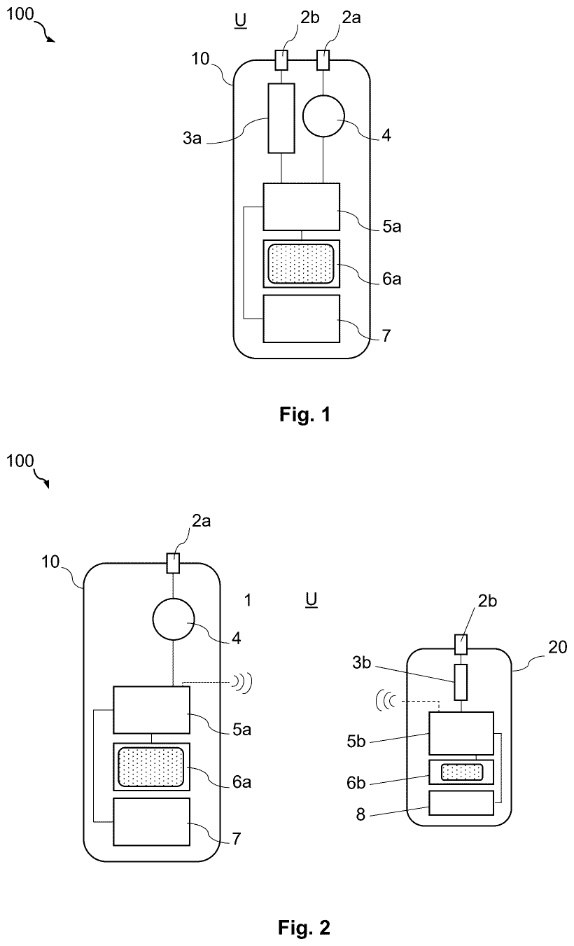

FIG. 2 shows a further exemplary embodiment of a schematically illustrated gas measuring system according to the disclosure,

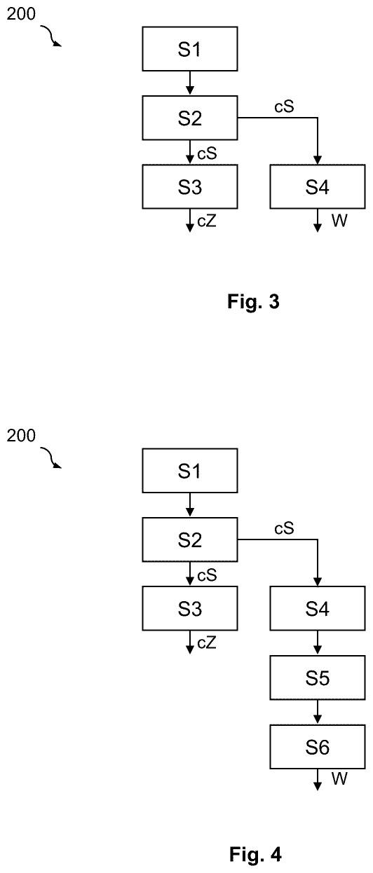

FIG. 3 shows a schematic flow diagram according to one exemplary embodiment of a method according to the disclosure,

FIG. 4 shows a schematic flow diagram according to a further exemplary embodiment of a method according to the disclosure,

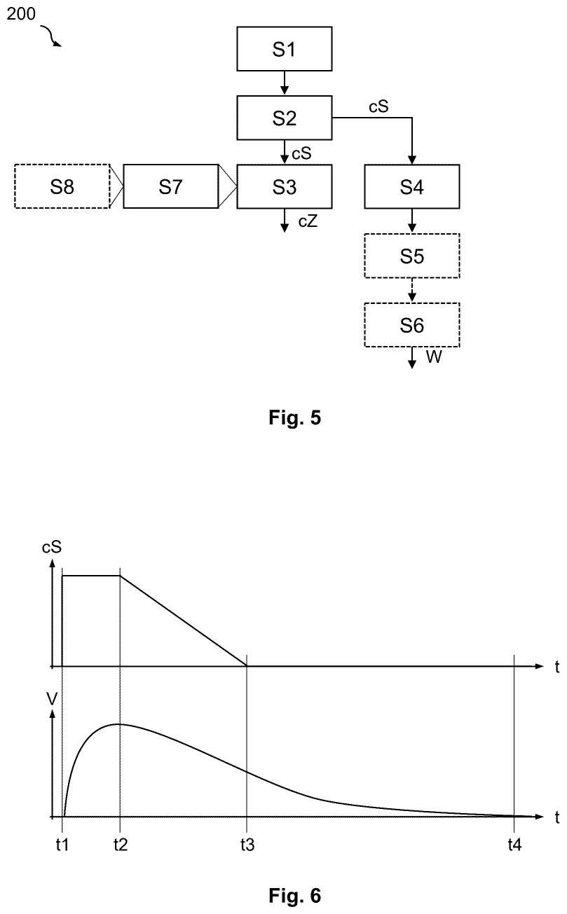

FIG. 5 shows a schematic flow diagram according to yet a further exemplary embodiment of a method according to the disclosure,

FIG. 6 shows example time curves of the concentration of harmful gas in a storage environment and of a state of poisoning.

According to the disclosure, a gas measuring system 100 is first provided. Two exemplary embodiments of gas measuring systems 100 according to the disclosure are shown in FIG. 1 and FIG. 2.

Each gas measuring system 100 according to the disclosure comprises a first gas sensor 3a, 3b, which is configured to provide a first measurement signal correlating with a concentration of a harmful gas cS in a storage environment U of the first gas sensor 3a, 3b. Each gas system 100 according to the disclosure further comprises a second gas sensor 4, which is configured to provide a second measurement signal correlating with a concentration of a target gas cZ in a measurement environment U of the second gas sensor 4. Furthermore, each gas measuring system 100 according to the disclosure has an evaluation unit 5a, 5b, which is configured to receive the first measurement signal and the second measurement signal and to carry out some or all of the steps S1-S8 of the method 200 according to the disclosure, which is described further herein.

Insofar as reference is only made to the gas measuring system 100 below, features and effects disclosed in this context apply to every possible embodiment of a gas measuring system 100 according to the disclosure.

The first gas sensor 3a, 3b, the second gas sensor 4 and the evaluation unit 5a, 5b can be designed as elements of a common device. This is the case in the exemplary embodiment according to FIG. 1, in which a gas measuring device 10 is provided, which comprises the components mentioned. Via corresponding openings 2a, 2b, the gas sensors 3a, 4 are in fluid communication with the environment U, which, depending on the operating state, can be the storage environment U or the measurement environment U. Via the openings 2a, 2b, the first gas sensor 3b and/or the second gas sensor 4 can operate using diffusion. Additionally or alternatively, the first gas sensor 3b and/or the second gas sensor 4 can be configured for active sampling via the openings 2a, 2b.

The measurement environment U can also be guided indirectly to the second gas sensor 4 through a sample supplying element such as a mouthpiece or a funnel, so that the measurement environment U can also be provided by a breathing gas sample of a test subject. For this purpose, the gas measuring device 10 can have an interface for receiving a sample supplying element, which can provide a fluidic connection between the opening 2a and a test subject's mouth.

It is also possible that the first gas sensor 3b, the second gas sensor 4 and the evaluation unit 5a, 5b can be elements of different devices of the gas measuring system 100. An example of such a case is shown in FIG. 2. In this exemplary embodiment, the second gas sensor 4 is provided in a gas measuring device 10, while the first gas sensor 3b and the evaluation unit 5b are elements of a monitoring device 20. The gas measuring device 10 in this exemplary embodiment also comprises an evaluation unit 5a so that the two evaluation units 5a, 5b can perform different or identical functions.

Each gas measuring device 10 can be designed as a breathalyzer 10, which is configured to determine a concentration of alcohol as a target gas in a measurement environment U of the breathalyzer 10, in particular in a breathing gas sample, wherein the breathalyzer 10 comprises the second gas sensor 4, as described.

The gas measuring system 100, in particular each component of the gas measuring system 100, may comprise further elements. In the exemplary embodiment according to FIG. 1, the gas measuring device 10, for example, further comprises an energy store 7 for supplying energy to the electrical and/or electronic components of the gas measuring device 10. Likewise, the gas measuring device 10 and the monitoring device 20 in the exemplary embodiment according to FIG. 2 comprise a corresponding energy store 7.

It is preferred and shown in the exemplary embodiments according to FIG. 1 and FIG. 2 that the gas measuring system 100, in particular the gas measuring device 10, for example the breathalyzer 10, and/or the monitoring device 20, can comprise an alarm unit 6a, 6b for optically and/or acoustically issuing the warning. The alarm unit 6a, 6b can, for example, be designed as a screen.

If the components of the gas measuring system 100 are not accommodated in a common gas measuring device 10, it is preferable for a data connection to be providable between the devices 10, 20 of the gas measuring system 100. This is indicated schematically in FIG. 2 as a radio connection. The same applies if the evaluation unit 5a, 5b is designed as a further component of the gas measuring system 100 that is physically separate from the devices 10, 20 (which is possible but not shown).

The first gas sensor 3a, 3b and the second gas sensor 4 can each be an electrochemical gas sensor.

If the gas measuring system 100 is realized by a plurality of devices 10, 20, it is preferable that only one of the devices 20 comprises the evaluation unit 5a, 5b. However, it is possible for each of the devices 10, 20 to have a (redundant or supplementary) evaluation unit 5a, 5b according to the disclosure.

Schematic flow diagrams of methods 200 according to the disclosure are shown in FIGS. 3, 4 and 5. Each method 200 according to the disclosure can be used to operate a gas measuring system 100 as described above.

Insofar as reference is only made hereinafter to the method 200, this equally applies to every possible embodiment of a method 200 according to the disclosure.

The method 200 comprises the step S1 of storing a gas measuring device 10 in a storage environment U in a first operating state. The first operating state can, for example, be a state in which a gas measurement by the second gas sensor 4 does not take place, but a gas measurement by the first gas sensor 3a, 3b takes place.

The method 200 comprises the step S2 of determining a concentration of a harmful gas in the storage environment U by means of an evaluation unit 5a, 5b. For this purpose, the evaluation unit 5a, 5b can use the measurement signal(s) of the first gas sensor 3a, 3b in (quasi-) real time or with a time delay, for example by retrieving temporarily stored data. Step S2 can be repeated, in particular repeatedly carried out periodically in the first operating state.

The method 200 comprises the step S3 of determining a concentration of the target gas cZ in a measurement environment U in a second operating state by means of the evaluation unit 5a, 5b, taking into account the concentration of the harmful gas cS, and outputting the concentration of the target gas cZ. Step S3 preferably takes place at a different time than step S2, i.e., after step S2. The second operating state can, for example, be a state in which the gas measurement by the second gas sensor 4 takes place.

The method 200 comprises, in addition or as an alternative to step S3, the step S4 of comparing information correlating with the concentration of the harmful gas cH with a predetermined threshold value by means of the evaluation unit 5a, 5b and issuing a warning in a third operating state if the correlating information exceeds the predetermined threshold value. Step S4 can be carried out substantially immediately if the predetermined threshold value is exceeded, or can be carried out at a later time, for example depending on further conditions. For example, step S4 can be carried out when the gas measuring device 10 is switched on, i.e., is transferred from the first operating state to the second operating state, in order to accordingly warn a user who is then certainly present.

The method 200 according to the disclosure can optionally further comprise steps S5 and S6, as shown in FIG. 4.

Step S5 is determining, by means of the evaluation unit 5a, 5b, whether the concentration and/or a quantity of the harmful gas has exceeded the predetermined threshold value.

Step S6 is issuing the warning if the concentration and/or the quantity of the harmful gas has exceeded the predetermined threshold value.

Step S6 can be carried out substantially at the same time as step S5 or at a later time, for example when the gas measuring device 10 is switched on, i.e., is transferred from the first operating state to the second operating state, in order to warn a user who is then certainly present.

For example, the determination of whether the quantity of the harmful gas has exceeded the predetermined threshold value can be made according to the following formula:

$${\sum }_{i = - N}^{0} a ( T ) \cdot cS ( t - i \cdot Δ t ) = \{ \begin{matrix}< g \to not exceeded \\ \geq g \to exceeded\end{matrix}$$

where g is the predetermined threshold value, cS is the concentration of the harmful gas, Δt is a sampling time of the measured values, N is the number of measured values to be taken into account, and a(T) is an optional weighting function that is dependent on the temperature T.

In a further example, the determination by the evaluation unit 5a, 5b can also be carried out using a recursive low-pass filter. In this example, the measured values for the concentration of the harmful gas cS serve as input of the low-pass filter, wherein the output signal of the low-pass filter is compared with the predetermined limit value.

It is preferred that, in the method 200 according to the disclosure, a first gas sensor 3a, 3b is provided for determining the concentration of the harmful gas cS in the storage environment U, wherein a second gas sensor 4 is provided for determining the concentration of the target gas cZ in the measurement environment U, as has been described in connection with the gas measuring system 100 according to the disclosure.

It is preferred and shown in the exemplary embodiment according to FIG. 5 that the step S3 comprises the step S7: correcting a sensitivity of the second gas sensor 4 by means of the evaluation unit 5a, 5b. In this respect, the step S7 can be provided as an additional step in each of the exemplary embodiments according to FIGS. 3 and 4.

In this respect, it is known to assign a measurement signal of a gas sensor to a corresponding value for a concentration of the measured gas (target gas and/or harmful gas) using a corresponding proportional factor, for example an adjustment factor. By adjusting the sensitivity, the proportional factor is effectively changed so that the measuring behavior of the second gas sensor 4 changed by the harmful gas can be taken into account accordingly.

It is preferred and also shown in the exemplary embodiment according to FIG. 5 that the step S7 can optionally comprise the further step S8: determining a current state of poisoning V and/or a current state of recovery of the second gas sensor 4 by means of the evaluation unit 5a, 5b.

For example, for correcting the sensitivity, both the state of poisoning V and the state of recovery can be modeled, that is, the dynamic behavior of poisoning and recovery. The determination of the state of poisoning V can be made using the concentration cS and/or quantity of the harmful gas.

The state of poisoning V can, for example, be modeled with a modified first-order model. The modification makes it possible to only take a progression of the poisoning into account if the current concentration and/or quantity of the harmful gas cS exceeds the current poisoning value V. As long as this condition is not met, recovery takes place, assuming that a time constant of the recovery τrec is smaller than a time constant of the poisoning cont.

For example, it is possible to model a coupled poisoning-recovery process using a first-order linear differential equation system, for example according to the following system:

$$τ cont \cdot \frac{\partial V ( t )}{\partial t} + V ( t ) = K \cdot c ( t ) for K \cdot c ( t ) \geq V ( t )$$

$$τ rec \cdot \frac{\partial V ( t )}{\partial t} + V ( t ) = K \cdot c ( t ) for K \cdot c ( t ) < V ( t )$$

where τcont represents the time constant for the poisoning process, τrec represents the time constant for the recovery process, V(t) represents the state of poisoning at time point t, c(t) represents the concentration of the harmful gas cS at time point t, and C represents a constant which defines the ratio between the degree of poisoning V(t) and the concentration of the harmful gas cS(t) in the steady state.

In the case that the concentration of the harmful gas cS is not determined continuously, but at discrete sampling time points with a time interval (sampling interval) Δt, discretization of the differential equation system and its numerical solution is possible. Discretization is possible, for example, using the forward Euler method or the Runge-Kutta method.

The model can be modified so that the time constant for the poisoning process τcont and the time constant for the recovery process τrec are also modeled depending on the current temperature.

By taking into account the current state of poisoning V and/or the current state of recovery, it is particularly advantageously possible to numerically correct the sensitivity of the second gas sensor 4. For example, a correction factor can be determined as a function of the current state of poisoning V and/or as a function of the current state of recovery, which correction factor can be multiplied by the proportional factor described above.

The determination of the correction factor as a function of the current state of poisoning V and/or as a function of the current state of recovery can, for example, be made empirically by setting certain stationary states of poisoning V in an experiment and determining the resulting effect on the sensitivity of the second gas sensor 4. The calculation rule thus obtained can then either be specified as an analytical expression or, for example, be stored in a look-up table for retrieval by the evaluation unit 5a, 5b.

In this respect, FIG. 6 shows an example of a curve of a concentration of harmful gas cS in a storage environment U over time t in a gas measuring system 100 according to the disclosure as well as a corresponding time curve of a state of poisoning V determined using the method 200 according to the disclosure.

The upper diagram in FIG. 6 shows that, in the example, the concentration of the harmful gas cS in a storage environment U increases abruptly at a time point t1 to a maximum, which is maintained until a time point t2, before it decreases again in a ramp-like manner to zero until a time point t3. The current poisoning V determined according to the method 200 according to the disclosure over time t is plotted in the lower diagram in FIG. 6. It can be seen that, starting from time point t1 until time point t2, the calculated poisoning V(t) increases continuously and then, beyond time point t3 until around time point t4, only slowly decreases to zero again.

### LIST OF REFERENCE SIGNS

2a, 2b Openings
3a, 3b First gas sensor
4 Second gas sensor
5a, 5b Evaluation unit
6a, 6b Alarm unit
7 Energy store
10 Gas measuring device, breathalyzer
20 Monitoring device
100 Gas measuring system
200 Method
cS Concentration of harmful gas
CZ Concentration of target gas
g Predetermined threshold value
S1, S2, . . . Method steps
t Time
t1, t2 Time points
U Environment, storage environment, measurement environment
V State of poisoning, poisoning value
W Warning

1-10. (canceled)

11. A method for operating a gas measuring system, comprising:
storing a gas measuring device in a storage environment in a first operating state;
determining a concentration of a harmful gas in the storage environment by means of an evaluation unit;
determining a concentration of a target gas in a measurement environment by means of the evaluation unit while the gas measuring device is in a second operating state;
performing at least one of:
outputting, based on the concentration of the harmful gas, the concentration of the target gas; or
comparing the concentration of the harmful gas to a predetermined threshold value by means of the evaluation unit, and
based on the comparing, issuing a warning while the gas measuring device is in a third operating state if the concentration of the harmful gas exceeds the predetermined threshold value; and
wherein the first operating state and the second operating state occur at different times.

12. The method of claim 11, wherein the determining the concentration of the harmful gas in the storage environment is repeated periodically.

13. The method of claim 12, further comprising:
determining, by means of the evaluation unit, whether at least one of the concentration of the harmful gas or a quantity of the harmful gas has exceeded the predetermined threshold value; and
issuing, based on a determination that the at least one of the concentration of the harmful gas or the quantity of the harmful gas has exceeded the predetermined threshold value, the warning.

14. The method of claim 11:
wherein the determining the concentration of the harmful gas in the storage environment is performed using a first gas sensor, and
wherein the determining the concentration of the target gas in the measurement environment is performed using a second gas sensor different from the first gas sensor.

15. The method of claim 14, wherein the determining the concentration of the target gas in the measurement environment by means of the evaluation unit and while the gas measuring device is in the second operating state comprises correcting a sensitivity of the second gas sensor.

16. The method of claim 15, wherein the correcting the sensitivity of the second gas sensor comprises determining, by means of the evaluation unit, at least one of:
a current state of poisoning of the second gas sensor; or
a current state of recovery of the second gas sensor.

17. The method of claim 15, wherein the correcting the sensitivity of the second gas sensor comprises receiving, by the evaluation unit and from the second gas sensor, one or more messages via a data connection.

18. The method of claim 14, wherein the first gas sensor and the second gas sensor are electrochemical gas sensors.

19. The method of claim 11, wherein the issuing the warning comprises outputting, to a user interface coupled to the evaluation unit, at least one of:
an acoustic alarm, or
an optical alarm.

20. The method of claim 11, wherein the comparing the concentration of the harmful gas to the predetermined threshold value is initiated based on the gas measuring device transitioning from the first operating state to the second operating state.

21. A gas measuring system comprising:
a first gas sensor, which is configured to provide a first measurement signal associated with a concentration of a harmful gas in a storage environment of the first gas sensor;
a second gas sensor, which is configured to provide a second measurement signal associated with a concentration of a target gas in a measurement environment of the second gas sensor; and
an evaluation unit, which is configured to:
receive the first measurement signal and the second measurement signal;
determine, based on the first measurement signal and the second measurement signal, the concentration of the harmful gas and the concentration of the target gas; and
perform at least one of:
outputting, based on the concentration of the harmful gas, the concentration of the target gas; or
comparing the concentration of the harmful gas to a predetermined threshold value, and
based on the comparing, issuing a warning if the concentration of the harmful gas exceeds the predetermined threshold value.

22. The gas measuring system of claim 21, further comprising:
a breathalyzer, which is configured to determine a concentration of alcohol as a second target gas in a measurement environment of the breathalyzer,
wherein the breathalyzer comprises the second gas sensor.

23. The gas measuring system according to claim 21, further comprising:
a monitoring device, which is configured to determine the concentration of the harmful gas in the storage environment,
wherein the monitoring device comprises the first gas sensor.

24. The gas measuring system of claim 21, wherein the first gas sensor comprises a breathalyzer and the second gas sensor comprises a monitoring device, and wherein at least one of the breathalyzer or the monitoring device comprises an alarm unit for issuing the warning.

25. The gas measuring system of claim 21, wherein the evaluation unit is further configured to:
determine whether at least one of the concentration of the harmful gas or a quantity of the harmful has exceeded the predetermined threshold value; and
issue, based on a determination that the at least one of the concentration of the harmful gas or the quantity of the harmful gas has exceeded the predetermined threshold value, the warning.

26. The gas measuring system of claim 21, wherein the evaluation unit is configured to determine the concentration of the target gas by correcting a sensitivity of the second gas sensor.

27. The gas measuring system of claim 26, wherein the evaluation unit is configured to correct the sensitivity of the gas sensor based on determining at least one of:
a current state of poisoning of the second gas sensor; or
a current state of recovery of the second gas sensor.

28. The gas measuring system of claim 21, wherein the first gas sensor and the second gas sensor are electrochemical gas sensors.

29. The gas measuring system of claim 21, wherein the evaluation unit is configured to issue the warning by outputting, to a user interface coupled to the evaluation unit, at least one of:
an acoustic alarm, or
an optical alarm.

30. The gas measuring system of claim 21, wherein the evaluation unit is configured to receive the second measurement signal via a wireless data connection between the evaluation unit and the second gas sensor.
